(19) United States
(12) Patent Application Publication
(10) Pub. No.: US 2024/0212787 A1
(43) Pub. Date: Jun. 27, 2024

(54) PROTEIN-TO-PROTEIN INTERFACE ANALYSIS

(71) Applicant: Genentech Inc., South San Francisco, CA (US)

(72) Inventors: Seth Facundo HARRIS, South San Francisco, CA (US); Kiran MUKHYALA, South San Francisco, CA (US)

(21) Appl. No.: 18/601,490

(22) Filed: Mar. 11, 2024

Related U.S. Application Data
(63) Continuation of application No. PCT/US2022/076443, filed on Sep. 14, 2022.
(60) Provisional application No. 63/244,168, filed on Sep. 14, 2021.

(51) Int. Cl. G16B 15/30; G06N 5/022; G16B 40/20

(52) U.S. Cl. CPC G16B 15/30; G06N 5/022; G16B 40/20

(57) ABSTRACT

A method for analyzing a family of protein-to-protein interfaces between a first protein group and a second protein group may include assigning, to each position within an aligned plurality of protein sequences from the first protein group and/or the second protein group, a family position identifier. One or more clusters of protein-to-protein interfaces within the family of protein-to-protein interfaces may be identified based on the family position identifier assigned to one or more positions included in each protein-to-protein interface in the family of protein-to-protein interfaces. One or more protein-to-protein interface properties may be determined for the one or more clusters of protein-to-protein interfaces. Related systems and computer program products are also provided.

### CROSS REFERENCE TO RELATED APPLICATION

This application claims priority to U.S. Provisional Application No. 63/244,168, entitled “PROTEIN-TO-PROTEIN INTERFACE ANALYSIS” and filed on Sep. 14, 2021, the disclosure of which incorporated herein by reference in its entirety.

### TECHNICAL FIELD

The subject matter described herein relates generally to the analysis of protein-to-protein interactions and more specifically to a framework for analyzing of protein-to-protein interactions across multiple protein families.

### INTRODUCTION

Proteins are responsible for many essential cellular functions including, for example, enzymatic reactions, transport of molecules, regulation and execution of a number of biological pathways, cell growth, proliferation, nutrient uptake, morphology, motility, intercellular communication, and/or the like. Although some proteins perform their functions independently, most biological activities require interactions between multiple proteins. For example, two or more protein molecules may establish physical contact driven by a variety of biochemical phenomena such as electrostatic forces, hydrogen bonding, Van der Waals forces, and hydrophobic effects. The resulting protein-to-protein interaction may be transient, in which case the proteins involved interact briefly and in a reversible manner. Alternatively, the protein-to-protein interaction may be stable and persist over a long period of time. Examples of protein-to-protein interactions include electron transfer, signal transduction, membrane transport, cell metabolism, and muscle contraction. Characterizing protein-to-protein interactions may provide critical insights into cellular function and biology.

### SUMMARY

Systems, methods, and articles of manufacture, including computer program products, are provided for analyzing of protein-to-protein interactions across multiple protein families. In one aspect, there is provided a system for analyzing protein-to-protein interfaces. The system may include at least one processor and at least one memory. The at least one memory may include program code that provides operations when executed by the at least one processor. The operations may include: identifying a family of protein-to-protein interfaces between a first protein group and a second protein group; assigning, to each position within an aligned plurality of protein sequences from the first protein group and/or the second protein group, a family position identifier; identifying, based at least on the family position identifier assigned to one or more positions included in each protein-to-protein interface in a family of protein-to-protein interfaces, one or more clusters of protein-to-protein interfaces within the family of protein-to-protein interfaces; and determining one or more protein-to-protein interface properties of the one or more clusters of protein-to-protein interfaces.

In another aspect, there is provided a method for analyzing protein-to-protein interfaces. The method may include: identifying a family of protein-to-protein interfaces between a first protein group and a second protein group; assigning, to each position within an aligned plurality of protein sequences from the first protein group and/or the second protein group, a family position identifier; identifying, based at least on the family position identifier assigned to one or more positions included in each protein-to-protein interface in a family of protein-to-protein interfaces, one or more clusters of protein-to-protein interfaces within the family of protein-to-protein interfaces; and determining one or more protein-to-protein interface properties of the one or more clusters of protein-to-protein interfaces.

In another aspect, there is provided a computer program product for analyzing protein-to-protein interfaces. The computer program product may include a non-transitory computer readable medium storing instructions that cause operations when executed by at least one data processor. The operations may include: identifying a family of protein-to-protein interfaces between a first protein group and a second protein group; assigning, to each position within an aligned plurality of protein sequences from the first protein group and/or the second protein group, a family position identifier; identifying, based at least on the family position identifier assigned to one or more positions included in each protein-to-protein interface in a family of protein-to-protein interfaces, one or more clusters of protein-to-protein interfaces within the family of protein-to-protein interfaces; and determining one or more protein-to-protein interface properties of the one or more clusters of protein-to-protein interfaces.

In some variations of the methods, systems, and non-transitory computer readable media, one or more of the following features can optionally be included in any feasible combination.

In some variations, the assigning of the family position identifier may include assigning, to a first position in a first protein sequence from the first protein group and a second position in a second protein sequence from the second protein group, a same family position identifier based at least on the first position being aligned with the second position.

In some variations, each cluster of protein-to-protein interfaces may include a plurality of protein-to-protein interfaces formed by interacting protein sequences that assume a same or similar docking pose.

In some variations, the one or more clusters of protein-to-protein interfaces may be identified by applying a hierarchical clustering.

In some variations, the one or more clusters of protein-to-protein interfaces may be further identified based on an amino acid residue occupying the one or more positions included in each protein-to-protein interface in the family of protein-to-protein interfaces.

In some variations, the one or more clusters of protein-to-protein interfaces may be further identified based on one or more of shape complementarity, frequency of amino acid residues, complementarity determining region (CDR) length, antigen-binding fragment (Fab) elbow angle, geometric feature, chemical feature, patch distribution, complementarity determining region (CDR) exposure, contact map, and biophysical property.

In some variations, the one or more clusters of protein-to-protein interfaces may be further identified by applying a filter imposing at least one selection criterion.

In some variations, the at least one selection criterion may include a minimum value and/or a maximum value associated with at least one of cluster size, interface area, solvation energy, stabilization energy, shape complementarity, complementarity determining region (CDR) exposure, and elbow angle.

In some variations, in response to a selection of a cluster of protein-to-protein interfaces from the one or more clusters of protein-to-protein interfaces, the one or more protein-to-protein interface properties for the selected cluster of protein-to-protein interfaces may be determined.

In some variations, a visual representation of a distribution of the one or more protein-to-protein interface properties across the selected cluster of protein-to-protein interfaces may be generated for display in a user interface.

In some variations, in response to a further selection of a protein-to-protein interface from the selected cluster of protein-to-protein interfaces, the one or more protein-to-protein interface properties for the selected protein-to-protein interface may be determined.

In some variations, a structural representation of the selected protein-to-protein interface may be generated for display in a user interface. The structural representation may include a first visual indicator identifying the selected protein-to-protein interface within a first protein structure and a second protein structure associated with the selected protein-to-protein interface.

In some variations, the structural representation of the selected protein-to-protein interface may further include a second visual indicator identifying, within the first protein structure and/or the second protein structure, one or more of a heavy chain, a light chain, a framework region (FR), and a complementarity determining region (CDR).

In some variations, a linear representation of the selected protein-to-protein interface may be generated for display in a user interface. The linear representation may include one or more visual indicators identifying, for each position within the selected protein-to-protein interface, an amino acid residue occupying the position, a type of bond, and at least one metric associated with the position.

In some variations, the at least one metric may include a buried surface area.

In some variations, in response to a further selection of a superset of protein-to-protein interfaces including the family of protein-to-protein interfaces, the one or more protein-to-protein interface properties for the selected superset of protein-to-protein interfaces may be determined.

In some variations, a visual representation of a distribution of the one or more protein-to-protein interface properties across the selected superset of protein-to-protein interfaces may be generated for display in a user interface.

In some variations, the visual representation may include a horizontal axis corresponding to a first protein-to-protein interface property and a vertical axis corresponding to a second protein-to-protein interface property.

In some variations, the visual representation may include one or more visual indicators identifying, for each protein-to-protein interface in the selected superset of protein-to-protein interfaces, an originating species and/or a family of the originating species.

In some variations, the one or more protein-to-protein interface properties may include shape complementarity, frequency of amino acid residues, complementarity determining region (CDR) length, antigen-binding fragment (Fab) elbow angle, geometric feature, chemical feature, patch distribution, complementarity determining region (CDR) exposure, contact map, and/or biophysical property.

In some variations, a visual representation of at least a portion of the one or more protein-to-protein interface properties may be generated for display in a user interface.

In some variations, the family of protein-to-protein interfaces may include antigen-binding fragment (Fab-Fab) interfaces, antigen binding fragment to antigen (Fab-Antigen) interfaces, or T-cell receptor to peptide-bound major histocompatibility complexes (TCR-pMHC) interfaces.

In some variations, each of the first protein group and the second protein group may include a family of proteins sharing one or more commonalities in evolutionary origin, function, sequence, and/or structure.

In some variations, each of the first protein group and the second protein group may include one of an antibody, a kinase, an antigen, a T-cell receptor (TCR), and a peptide-bound major histocompatibility complex (pMHC).

In some variations, labeled training data for training a machine learning model to identify protein sequences having the one or more protein-to-protein interface properties may be generated based at least on the one or more protein-to-protein interface properties.

In some variations, a starting protein sequence providing a basis upon which a machine learning model generates one or more additional protein sequences may be generated based at least on the one or more protein-to-protein interface properties.

In some variations, one or more protein-to-protein interfaces from the family of protein-to-protein interfaces may be identified based at least on the one or more protein-to-protein interface properties. One or more mutations to increase a stability of a complex having the one or more protein-to-protein interface may be applied to the one or more protein-to-protein interfaces.

In some variations, the one or more mutations may improve one or more of crystal packing, hydrogen bond interactions, and cysteine scanning at the one or more protein-to-protein interface.

In some variations, one or more positions within a protein sequence that can be modified when designing the protein sequence to exhibit one or more desirable properties may be identified based at least on the one or more protein-to-protein interface properties.

In some variations, one or more positions within a protein sequence that remain fixed when designing the protein sequence to exhibit one or more desirable properties may be identified based at least on the one or more protein-to-protein interface properties.

In some variations, an amino acid residue that is most likely or least likely to occupy at least one position within a protein sequence when designing the protein sequence to exhibit one or more desirable properties may be identified based at least on the one or more protein-to-protein interface properties.

In some variations, one or more known patterns of amino acid residues present in the first protein group and/or the second protein group may be validated based at least on the one or more protein-to-protein interface properties.

In some variations, the family of protein-to-protein interfaces may be identified based on one or more user inputs selecting the family of protein-to-protein interfaces or the first protein group and the second protein group.

In some variations, a plurality of protein sequences from the first protein group and/or the second protein group may be aligned.

In some variations, the plurality of protein sequences may be aligned by applying one or more of dynamic programming, progressive alignment, hierarchical alignment, iterative alignment, motif finding, and Hidden Markov models.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the analysis of protein-to-protein interactions, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations.

In the drawings,

FIG. 1 depicts a system diagram illustrating an example of a protein analysis system, in accordance with some example embodiments;

FIG. 2 depicts a schematic diagram illustrating an example of aligned protein sequences from a protein family, in accordance with some example embodiments;

FIG. 3 depicts a screenshot of an example of a user interface, in accordance with some example embodiments;

FIG. 4 depicts a screenshot of another example of a user interface, in accordance with some example embodiments;

FIG. 5A depicts a screenshot of another example of a user interface, in accordance with some example embodiments;

FIG. 5B depicts a screenshot of another example of a user interface, in accordance with some example embodiments;

FIG. 5C depicts a screenshot of another example of a user interface, in accordance with some example embodiments;

FIG. 6A depicts a screenshot of another example of a user interface, in accordance with some example embodiments;

FIG. 6B depicts a screenshot of another example of a user interface, in accordance with some example embodiments;

FIG. 6C depicts a screenshot of another example of a user interface, in accordance with some example embodiments;

FIG. 6D depicts a screenshot of another example of a user interface, in accordance with some example embodiments;

FIG. 7 depicts a flowchart illustrating an example of a process for analyzing protein-to-protein interfaces, in accordance with some example embodiments; and

FIG. 8 depicts a block diagram illustrating an example of a computing system, in accordance with some example embodiments.

When practical, similar reference numbers denote similar structures, features, or elements.

### DETAILED DESCRIPTION

Analysis of protein-to-protein interactions, which underpin numerous essential cellular functions, may provide critical insights into cellular function and biology. Although the availability of protein structural data facilitates the study of protein-to-protein interfaces, conventional software tools are highly specialized and configured for specific protein families of interest. For example, while one analytical software may support the analysis of antibodies, a different analytical software may be required for the analysis of kinases. That a different software tool is required for the analysis of each protein family may complicate and thwart research efforts, especially when analysis across different protein families share common objectives such as site-directed mutagenesis and rational protein design. Moreover, conventional software tools are limited to the analysis of individual pairs of interacting proteins and are thus incapable of providing insights into the protein-to-protein interactions that exist in the context of entire protein families.

As such, in some example embodiments, an analysis engine may be configured to support a generalized analysis of protein-to-protein interfaces where protein-to-protein interactions, such as bindings, occur as between protein sequences from within a single protein family of interest or protein sequences from different protein families of interest. For example, the analysis engine may identify and characterize protein interfaces from structural data associated with biological interfaces identified and derived through empirical means as well as mathematically synthesized interfaces characterized by mathematical operations (e.g., crystallographic symmetry, symmetry operators, and/or the like), computational models, and/or the like. Conventional approaches to protein analysis are limited to biological structures or interfaces that have been realized in the laboratory. Contrastingly, various implementations of the analysis engine described herein also support the analysis of mathematically synthesized interfaces, thus lending insights into structures and interfaces across the spectrum of those that have been derived in a laboratory and those that exist purely in the mathematically synthesized realm.

As used herein, the term “protein-to-protein interface” refers to one or more portions of a protein sequence (e.g., one or more subsequence of amino acid residues within the protein sequence) that interacts with another protein sequence when the two protein sequences interact. Examples of protein-to-protein interfaces include V-H interface present in antigen-binding fragment (Fab) and antigen complexes, the antigen-binding fragment (Fab) and antigen interface, the T-cell receptor (TCR) peptide interface in T-cell receptor and peptide-bound major histocompatibility complexes (TCR-pMHC), and/or the like. A generalized analysis of protein-to-protein interactions between protein sequences from the same protein family and/or different protein families may provide a variety insights in the context of entire protein families including, for example, commonalities and differences in the constituent amino acid residues, the positions, the types of bonds, the size, and/or the location of various protein-to-protein interfaces that exist at the family level.

In some example embodiments, the analysis engine may perform an analytical workflow that includes aligning proteins sequence in each protein family, applying a universal numbering scheme for referencing residue positions within each protein sequence, identifying the amino acid residues at the protein-to-protein interface where protein-to-protein interaction takes place, measuring one or more properties of the interface, and applying a variety of computational analysis. In some instances, in addition to identifying the amino acid residues that are present at the protein-to-protein interface, the analysis engine may calculate additional interface properties such as shape complementarity, amino acid frequencies, complementarity determining region (CDR) lengths, antigen-binding fragment (Fab) elbow angles, interaction fingerprint (e.g., geometric features, chemical features), patch distribution, complementarity determining region (CDR) exposure, geometric features, contact maps, biophysical properties, and/or the like.

In some example embodiments, the analysis engine may align the protein sequences in each family of interacting proteins by applying a variety of sequence alignment techniques such as dynamic programming, progressive or hierarchical alignment, iterative alignment, motif finding, and Hidden Markov models. Furthermore, the analysis engine may apply, to the aligned protein sequences within each family of interacting proteins, a universal numbering scheme. This may include assigning, to the individual positions within each aligned protein sequence of a protein family, a family position identifier that is consistent across the aligned protein sequences within the protein family. For example, where a first position from a first protein sequence is aligned with a second position from a second protein sequence upon aligning the protein sequences from the protein family, the same family position identifier may be assigned to the first position and the second position. Accordingly, the same family position identifier may reference the first position in the first protein sequence as well as the second position in the second protein sequence. The uniformity associated with the universal numbering scheme may be especially advantageous for performing a comparative analysis of the protein-to-protein interfaces that exist within a protein family.

In some example embodiments, the analysis engine may perform a variety of computational analysis based on one or more properties of protein-to-protein interfaces that exist between two or more molecules. As used herein, a protein-to-protein interface may exist between two protein molecules originating from a same protein family or different protein families. Alternatively, in some cases, a protein-to-protein interface may also exist between a non-protein molecule, such as a small molecule, nucleic acid, polysaccharide, or glycolipid, and a protein molecule from a particular protein family. The analysis engine may perform the computational analysis in order to determine the commonalities and/or differences that exist within a family of protein-to-protein interfaces such as the composition of the protein-to-protein interfaces (e.g., the amino acid residues forming each protein-to-protein interface), the types of bonds forming the protein-to-protein interfaces, the location of the protein-to-protein interfaces (e.g., the positions and/or regions in the protein sequences included in each protein-to-protein interface), the size of the protein-to-protein interfaces (e.g., the quantity of amino acid residues included in each protein-to-protein interface), and/or the like. Examples of such computational analysis may include dimensionality reduction (e.g., principal component analysis (PCA), uniform manifold approximation and projection (UMAP), t-distributed stochastic neighbor embedding (t-SNE), and/or the like), a cluster analysis (e.g., connectivity clustering, centroid clustering, distribution clustering, density clustering, hierarchical clustering, and/or the like), computing a similarity coefficient (e.g., Jaccard index and/or the like), and/or the like.

In some example embodiments, the analysis engine may perform a cluster analysis, such as a hierarchical cluster analysis, of a family of protein-to-protein interfaces that exist within a single protein family (e.g., when a non-protein molecule binds with a protein molecule from the protein family) and/or between two or more protein families of interest (e.g., when a first protein molecule from a first protein family binds with a second protein molecule from a second protein family). The analysis engine may perform the cluster analysis to identify groups of similar protein-to-protein interfaces across a variety of protein-to-protein interface properties. For example, the analysis engine may cluster the protein-to-protein interfaces based on the positions within each protein sequence, as referenced by the corresponding family position identifiers, that are involved in the interactions. Doing so may identify groups of protein-to-protein interfaces where the interacting protein sequences assume a same or similar pose (e.g., docking pose). Alternatively and/or additionally, the analysis engine may cluster the protein-to-protein interfaces based on the amino acid residues from each protein sequence that are involved in the interactions. It should be appreciated that the clustering of protein-to-protein interfaces may also be performed based on other characteristics of the protein-to-protein interface including, for example, shape complementarity, amino acid frequencies, complementarity determining region (CDR) lengths, antigen-binding fragment (Fab) elbow angles, interaction fingerprint (e.g., geometric features, chemical features), patch distribution, complementarity determining region (CDR) exposure, geometric features, contact maps, biophysical properties (e.g., electrostatics, hydrophilicity, hydrophobicity, molecular size) of the amino acid residues, and/or the like.

In some example embodiments, the analysis engine may perform, based on the results of the analytical workflow, a variety of downstream tasks including, for example, mining the antigen-binding fragment (Fab) for stability engineering, identifying sites for antigen-binding fragment (Fab) design at the VH/VL interface, validating and generating new insights for known patterns in the variable regions of an antibody, exploring the T-cell receptor and peptide-bound major histocompatibility complexes (TCR-pMHC) interface, generating training samples for machine learning, and/or the like. For example, in some instances, the analysis engine may identify, based at least on the results of the analytical workflow, one or more positions within a protein sequence that may be modified or must remain fixed when designing the protein sequence. Alternatively and/or additionally, the analysis engine may identify, based at least on the results of the analytical workflow, an amino acid residue that is most likely or least likely to occupy one or more positions within a protein sequence when designing the protein sequence.

In some cases, the analysis engine may generate, based on the results of the workflow, training data for training a machine learning model to recognize the characteristics of an optimal protein-to-protein interface. For example, the training data may include, for each protein sequence, one or more ground truth labels identifying various characteristics of the protein-to-protein interface (e.g., positions, amino acid residues, size, types of bonds, and/or the like). Accordingly, the machine learning model may be trained, based on the characteristics of known protein-to-protein interfaces, to determine the affinity between a protein and a ligand such as between an antibody and an antigen, a T-cell receptor (TCR) and a peptide major histocompatibility complex (pMHC), and/or the like. Alternatively and/or additionally, the machine learning model may be trained to determine, based on the characteristics of the protein-to-protein interfaces, certain properties of the interacting proteins. For instance, the machine learning model may determine, based at least on the region of interaction between antibodies (e.g., the prevalence of head-to-head interactions), the viscosity of the antibodies.

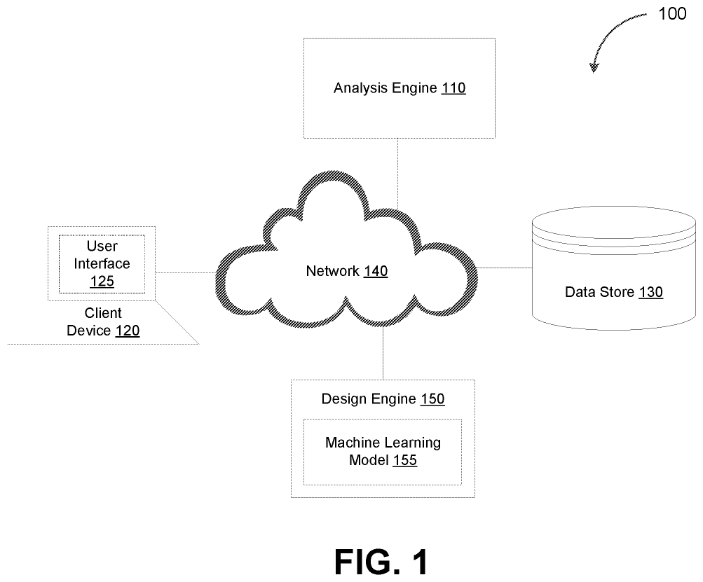

FIG. 1 depicts a system diagram illustrating an example of a protein analysis system 100, in accordance with some example embodiments. Referring to FIG. 1, the protein analysis system 100 may include an analysis engine 110, a client device 120, and a data store 130. As shown in FIG. 1, the analysis engine 110, the client device 120, and the data store 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The client device 120 may be a processor-based device including, for example, a workstation, a desktop computer, a laptop computer, a smartphone, a tablet computer, a wearable apparatus, and/or the like. The data store 130 may be a database including, for example, a relational database, a graph database, an in-memory database, a non-SQL (NoSQL) database, and/or the like.

In some example embodiments, the analysis engine 110 may be configured to support a generalized analysis of the protein-to-protein interfaces that exist between protein sequences from a single protein family and/or different protein families of interest. As noted, examples of protein-to-protein interfaces include the V-H interface present in antigen-binding fragment (Fab) and antigen complexes, the antigen-binding fragment (Fab) and antigen interface, the T-cell receptor (TCR) peptide interface in T-cell receptor and peptide-bound major histocompatibility complexes (TCR-pMHC), and/or the like. To support the generalized analysis of protein-to-protein interfaces, the analysis engine 110 may perform an analytical workflow that includes aligning proteins sequence in each protein family, applying a universal numbering scheme for referencing residue positions within each protein sequence, identifying the amino acid residues at the protein-to-protein interface where protein-to-protein interaction takes place, measuring one or more properties of the interface, and applying a variety of computational analysis. In the example of the protein analysis system 100 shown in FIG. 1, the data store 130 may store data associated with a variety of protein-to-protein interfaces. For example, for a family of protein-to-protein interfaces between a first protein family and a second protein family, the data store 130 may include data corresponding to various protein-to-protein interface properties such as the positions, amino acid residues, size, area, solvation energy, stabilization energy, shape complementarity, complementarity determining region (CDR) exposure, and elbow angles associated with each protein-to-protein interface. Accordingly, at least a portion of the aforementioned analytical workflow may be performed based on the data in the data store 130.

In some example embodiments, the analysis engine 110 may align the protein sequences in a protein family as part of the analytical workflow for a generalized analysis protein-to-protein interfaces. As used herein, the term “protein family” may refer to a group of proteins that share commonalities in one or more of an evolutionary origin, function, sequence, and/or structure. Examples of protein families include regulatory protein gene families (e.g., 14-3-3 protein family, Achaete-scute complex, forkhead box proteins, DLX gene family, Hox gene family, POU family, Krüppel-type zinc finger (ZNF), MADS-box gene family, NOTCH2NL, P300-CBP coactivator family, SOX gene family), immune system proteins (e.g., immunoglobulin superfamily, major histocompatibility complex (MHC)), motor proteins (e.g., dynein, kinesin, myosin), signal transducing proteins (e.g., G-proteins, MAP kinase, olfactory receptor, peroxiredoxin, receptor tyrosine kinases), and transporters (e.g., ABC transporters, antiporter, aquaporins). Additional examples of protein families include ATCase/OTCase family, bacterial potassium transporter, DHH phosphatase family, expansin gene family, fibroblast growth factors (FGF), fibroblast growth factor receptors (FGFR), FH2 protein (formin) gene family, FGD (FYVE, RhoGEF, and PH domain containing) family, heat shock proteins, ion channels, membrane spanning 4A, peroxin, protocadherin gene family, roundabout family, and SNARE family.

Aligning the protein sequences in a protein family may include arranging the protein sequences based on regions of similarities (e.g., same or similar subsequences of amino acid residues) present across multiple protein sequences, which are attributable to the functional, structural, and/or evolutionary relationships between the protein sequences within the protein family. It should be appreciated that the analysis engine 110 may apply a variety of sequence alignment techniques including, for example, dynamic programming, progressive or hierarchical alignment, iterative alignment, motif finding, Hidden Markov models, and/or the like. In some cases, the analysis engine 110 may align the protein sequences in a protein family based on an existing numbering scheme associated with the protein family (e.g., generic G protein-coupled receptors (GPCR) residue numbering). However, the existing numbering scheme associated with the protein family does not supplant the universal numbering scheme described in more detail below.

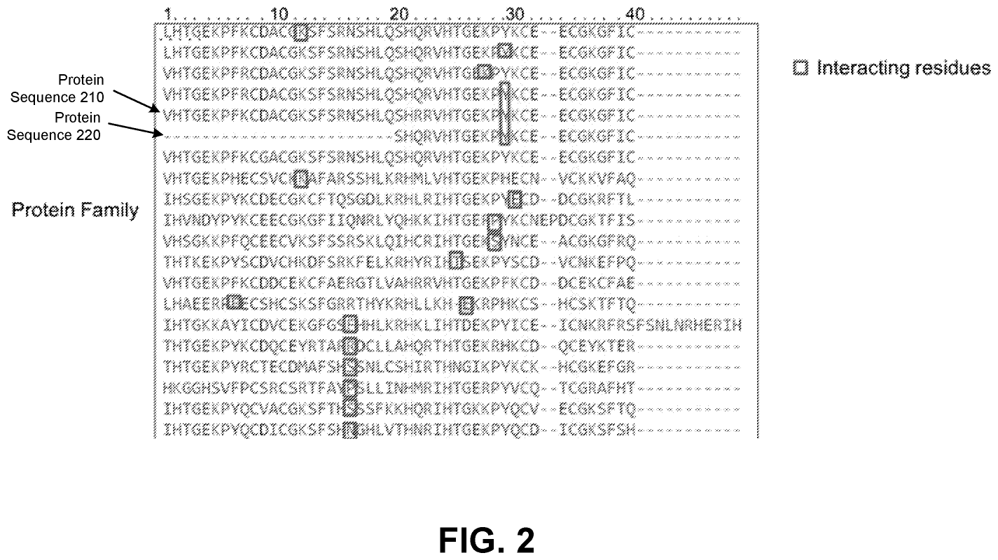

To further illustrate, FIG. 2 depicts a schematic diagram illustrating an example of at least a portion of the protein sequences from a protein family, which have been aligned based on regions of similarities (e.g., same or similar subsequences of amino acid residues) present across protein sequences. In some example embodiments, upon aligning the protein sequences in a protein family, the analysis engine 110 may continue the analytical workflow by at least applying a universal numbering scheme for referencing residue positions within each protein sequence. For example, the analysis engine 110 may apply the universal numbering scheme by assigning, to the positions in each aligned protein sequence in the protein family, a family position identifier that is consistent across the aligned protein sequences within the protein family. To enable subsequent comparative analysis of the protein-to-protein interfaces that exist within the protein family, the same family position identifier may be assigned to the positions within the aligned protein sequences. For instance, the same family position identifier may be assigned to a first position in a first protein sequence that is aligned with a second position within a second protein sequence even if the first position and the second position differ. Doing so may enable, for example, an identification of the family positions mostly frequently occupied by interacting residues as well as an analysis of the types of interacting residues. Interacting residues, which refer to amino acid residues forming the protein-to-protein interface, may be identified by applying a variety of tools and techniques including, for example, Protein Interface Structures and Assemblies (PISA).

An example application of the universal numbering scheme is shown in FIG. 2 where the family position identifier 29 is assigned to position 29 of a first protein sequence 210 and position 12 of a second protein sequence 220. In the example shown in FIG. 2, position 29 of the first protein sequence 210 and position 12 of the second protein sequence 220 are occupied by an interacting amino acid residue (e.g., tyrosine (Y)). As such, position 29 of the first protein sequence 210 and position 12 of the second protein sequence 220 form a part of the protein-to-protein interface between protein sequences of the protein family shown in FIG. 2 and those from another protein family. As described in more detail below, the family position identifier 29 assigned to position 29 of the first protein sequence 210 and position 12 of the second protein sequence 220 may be used to perform various comparative analysis of the protein-to-protein interfaces that exist between the two protein families.

Referring again to FIG. 1, the analysis engine 110 may perform the analytical workflow based on one or more user inputs received from the client device 120. For example, as shown in FIG. 1, the analysis engine 110 may generate, for display at the client device 120, a user interface 125. The one or more user inputs, which may be received via the user interface 125, may specify one or more subsets of data included in the data store 130 such as one or more protein families of interest, one or more protein-to-protein interface properties, and/or the like. For instance, in some cases, the one or more user inputs may select a particular family of protein-to-protein interfaces of interest for generalized analysis on a family level before selecting a subset of protein-to-protein interfaces from within the family for further analysis. Moreover, in some example embodiments, the analysis engine 110 may generate, for display as a part of the user interface 125 at the client device 120, one or more visual representations of at least a portion of the results of the analytical workflow performed by the analysis engine 110. The user interface 125 may be interactive such that the types of the visual representations and the contents presented therein may be updated in response to the one or more user inputs.

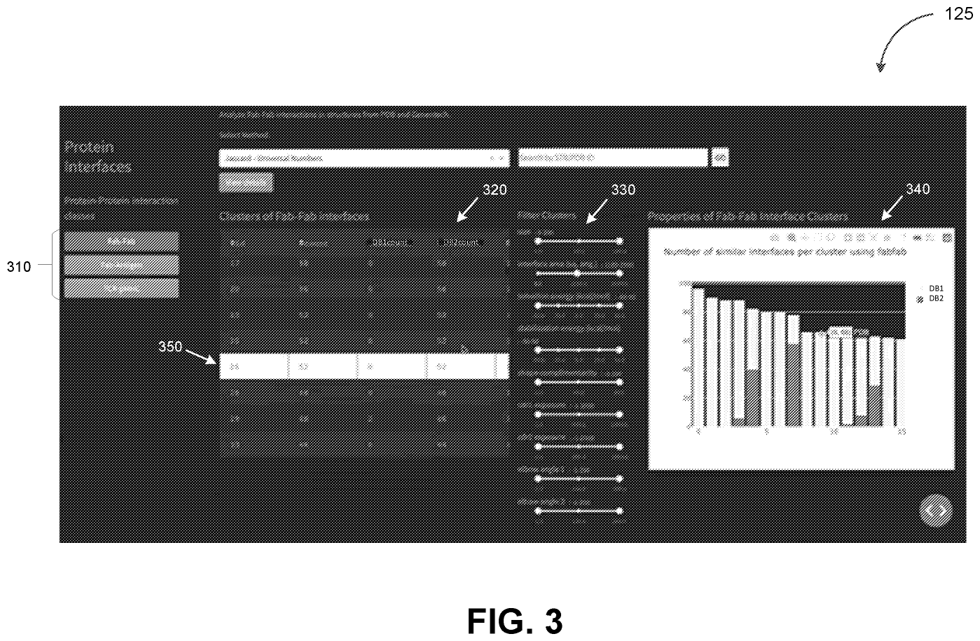

To further illustrate, FIG. 3 depicts a screenshot of an example of the user interface 125, in accordance with some example embodiments. The example of the user interface 125 shown in FIG. 3 includes one or more first input controls 310 for receiving one or more user inputs selecting a family (or class) of protein-to-protein interfaces, such as antigen-binding fragment to antigen-binding fragment (Fab-Fab) interfaces, antigen binding fragment to antigen (Fab-Antigen) interfaces, and T-cell receptor to peptide-bound major histocompatibility complexes (TCR-pMHC) interfaces, for analysis by the analysis engine 110. Upon receiving the one or more user inputs selecting a family (or class) of protein-to-protein for analysis, the analysis engine 110 may perform at least a portion of the analytical workflow and update the user interface 125 to display at least a portion of the results of the analytical workflow. For instance, the example of the user interface 125 shown in FIG. 3 includes the results of a cluster analysis performed on the family of antigen-binding fragment to antigen-binding fragment (Fab-Fab) interfaces.

In some example embodiments, the analysis engine 110 may perform a cluster analysis, such as a hierarchical cluster analysis, of various protein-to-protein interfaces. As noted, protein-to-protein interfaces may exists within a single protein family (e.g., when a non-protein molecule binds with a protein molecule from the protein family) or between two or more protein families (e.g., when a first protein molecule from a first protein family binds with a second protein molecule from a second protein family). In the example shown in FIG. 3, the cluster analysis may be performed on the protein-to-protein interfaces that exist between two families antigen-binding fragments (Fabs). The analysis engine 110 may perform the cluster analysis to identify groups of similar antigen-binding fragment to antigen-binding fragment (Fab-Fab) interfaces across a variety of protein-to-protein interface properties. For instance, this cluster analysis may be performed based on various protein-to-protein interface properties including, for example, positions involved (e.g., as referenced by the family position identifier), shape complementarity, amino acid frequencies, complementarity determining region (CDR) lengths, antigen-binding fragment (Fab) elbow angles, interaction fingerprint (e.g., geometric features, chemical features), patch distribution, complementarity determining region (CDR) exposure, geometric features, contact maps, biophysical properties (e.g., electrostatics, hydrophilicity, hydrophobicity, molecular size) of the amino acid residues, and/or the like.

In one example, the analysis engine 110 may cluster the protein-to-protein interfaces (e.g., the antigen-binding fragment to antigen-binding fragment (Fab-Fab) interfaces) based on the positions within each protein sequence, as referenced by the corresponding family position identifiers, that are involved in the interactions therebetween. Doing so may identify clusters of protein-to-protein interfaces in which the corresponding protein sequences assume a similar pose (e.g., docking pose) during interaction. For instance, the example of the user interface 125 shown in FIG. 3 includes a table 320 enumerating the different clusters of protein-to-protein interfaces identified within a corresponding family of protein-to-protein interfaces (e.g., antigen-binding fragment to antigen-binding fragment (Fab-Fab) interfaces). As shown in FIG. 3, the table 320 may include various statistics for the protein-to-protein interfaces in each cluster of protein-to-protein interfaces including, for example, quantity, data source (e.g., a first database DB1, a second database DB2, and/or the like), average size (e.g., measured in the quantity of constituent amino acid residues), average area, average solvation energy, average stabilization energy, shape complementarity, average magnitude of complementarity determining region (CDR) exposure, average elbow angles, and/or the like. The statistics included in the table 320 may enable an identification of the most common protein-to-protein interfaces (e.g., antigen-binding fragment to antigen-binding fragment (Fab-Fab) interfaces) within that family of protein-to-protein interfaces as well as various corresponding characteristics.

In addition to the table 320, the user interface 125 also includes a graph 340 providing a visual representation of at least a portion of the data shown in the table 320. In the example shown in FIG. 3, the graph 340 is a bar graph in which each bar corresponds to one of the clusters enumerated in the table 320. The dimensions of each bar in the graph 340 may correspond to a size of the corresponding cluster (e.g., the quantity of protein-to-protein interfaces included in the cluster). Moreover, the graph 340 may include a visual indication of the proportion of protein-to-protein interfaces (e.g., antigen-binding fragment to antigen-binding fragment (Fab-Fab) interfaces) originating from each data source (e.g., the first database DB1, the second database DB2, and/or the like). For example, for each bar in the example of the graph 340 shown in FIG. 3, a first portion of the bar corresponding to protein-to-protein interfaces from the first database DB1 may be rendered in a first color and sized to correspond to the quantity of the protein-to-protein interfaces from the first database DB1. Meanwhile, a second portion of the bar corresponding to the protein-to-protein interfaces from the second database DB2 may be rendered in a second color and sized to correspond to the quantity of protein-to-protein interfaces from the second database DB2.

Referring again to FIG. 3, the user interface 125 may include one or more second input controls 330 for adjusting one or more filters imposing at least one corresponding selection criterion. The one or more filters may therefore be applied to identify a subset of the clusters of protein-to-protein interfaces (e.g., antigen-binding fragment to antigen-binding fragment (Fab-Fab) interface) whose properties satisfy the selection criteria. Examples of filters shown in FIG. 3 include the size of a protein-to-protein interface (e.g., as measured in the quantity of constituent amino acid residues), area of the protein-to-protein interface, solvation energy, stabilization energy, shape complementarity, complementarity determining region (CDR) exposure, and elbow angles.

Adjustments made to a filter via the one or more second input controls 330 may set one or more thresholds (e.g., a maximum value, a minimum value, and/or the like) to a corresponding protein-to-protein interface property. Accordingly, the analysis engine 110 may identify one or more clusters of the protein-to-protein interfaces (e.g., antigen-binding fragment to antigen-binding fragment (Fab-Fab) interfaces) satisfying the one or more thresholds. For example, where the user inputs received via the one or more second input controls 330 set one or more thresholds with respect to cluster size (e.g., a maximum quantity and/or a minimum quantity of protein-to-protein interfaces in a cluster), the analysis engine 110 may identify one or more clusters of protein interfaces (e.g., antigen-binding fragment to antigen-binding fragment (Fab-Fab) interfaces) whose size satisfies the one or more thresholds. In some cases, the analysis engine 110 may update the table 320 and/or the graph 340 to include the clusters that satisfy the one or more thresholds set via the one or more second input controls 330 and exclude the clusters that fail to satisfy the one or more thresholds. For instance, in response to the user inputs setting the one or more thresholds with respect to cluster size (e.g., a maximum quantity and/or a minimum quantity of protein-to-protein interfaces in a cluster), the analysis engine 110 may update the table 320 and/or the graph 340 to include the clusters of protein-to-protein interfaces (e.g., antigen-binding fragment to antigen-binding fragment (Fab-Fab) interfaces) whose size satisfy the one or more thresholds and exclude the clusters whose size does not satisfy the one or more clusters.

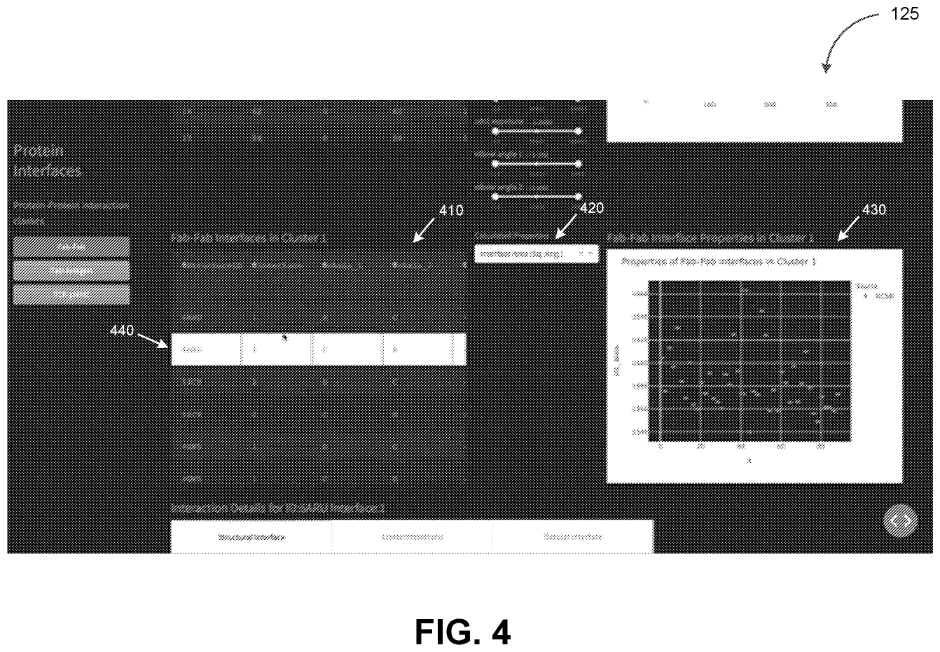

In some example embodiments, the analysis engine 110 may support the generalized analysis of protein-to-protein interfaces on a family level as well as a more granular analysis of certain subsets of protein-to-protein interfaces within the family of protein-to-protein interfaces. Returning again to the example shown in FIG. 3, the analysis engine 110 may receive one or more user inputs selecting one of the clusters of protein-to-protein interfaces (e.g., antigen-binding fragment to antigen-binding fragment (Fab-Fab) interfaces) for further analysis. For instance, the one or more user inputs may include a selection of the cluster 350 of protein-to-protein interfaces displayed in the table 320 for further, more granular analysis on the cluster level. The cluster 350 may be selected by selecting a corresponding row from the table 320 and/or a corresponding visual indicator (e.g., vertical bar) from the graph 340. In some cases, the cluster 350 may be selected for more granular analysis based on one or more of its properties, such as an unusually large number of constituent protein-to-protein interfaces, displayed in the table 320 and/or the graph 340. One example of this more granular, cluster level analysis is shown in FIG. 4, which depicts another example of the user interface 125 updated to display at least a portion of the results of the analytical workflow associated with the cluster 350 of protein-to-protein interfaces.

Referring now to FIG. 4, the example of the user interface 125 shown in FIG. 4 includes a table 410 enumerating the individual protein-to-protein interfaces included in the cluster 350 as well as the corresponding protein-to-protein interface properties. For example, the table 410 may include the quantity and identities of the amino acid residues included in each protein-to-protein interface. Furthermore, the example of the user interface 125 shown in FIG. 4 includes a graph 430 providing a visual representation of one or more protein-to-protein interface properties selected via the input control 420. For instance, in the example shown in FIG. 4, the graph 430 is a scatter plot showing the distribution of the interface area of the protein-to-protein interfaces included in the cluster 350. Other examples of protein-to-protein interface properties that may be selected via the input control 420 and displayed as a part of the graph 430 include the size of a protein-to-protein interface (e.g., as measured in the quantity of constituent amino acid residues), solvation energy, stabilization energy, shape complementarity, complementarity determining region (CDR) exposure (e.g., of each protein sequence involved in the interaction), and elbow angles (e.g., of each protein sequence involved in the interaction.

In addition to the cluster level analysis shown in FIG. 4, the analysis engine 110 may support an even more granular analysis at the individual protein-to-protein interface level. For example, FIG. 4 shows that the analysis engine 110 may receive one or more user inputs selecting a specific protein-to-protein interface from the cluster 350, such as the protein-to-protein interface 440, for further analysis. In some cases, the protein-to-protein interface 440 may be selected by selecting (e.g., highlighting) a corresponding row in the table 410. Alternatively and/or additionally, the protein-to-protein interface 440 may be selected by selecting a corresponding visual indicator in the graph 430. In some cases, the protein-to-protein interface 440 may be selected for more granular analysis based on one or more of its properties such as, in the case shown in FIG. 4, an unusually high interface area. For example, the analysis engine 110 may apply one or more adjustable filters, such as the size of a protein-to-protein interface (e.g., as measured in the quantity of constituent amino acid residues), area of the protein-to-protein interface, solvation energy, stabilization energy, shape complementarity, complementarity determining region (CDR) exposure, and elbow angles, in order to provide a more refined selection of protein-to-protein interfaces from the cluster 350.

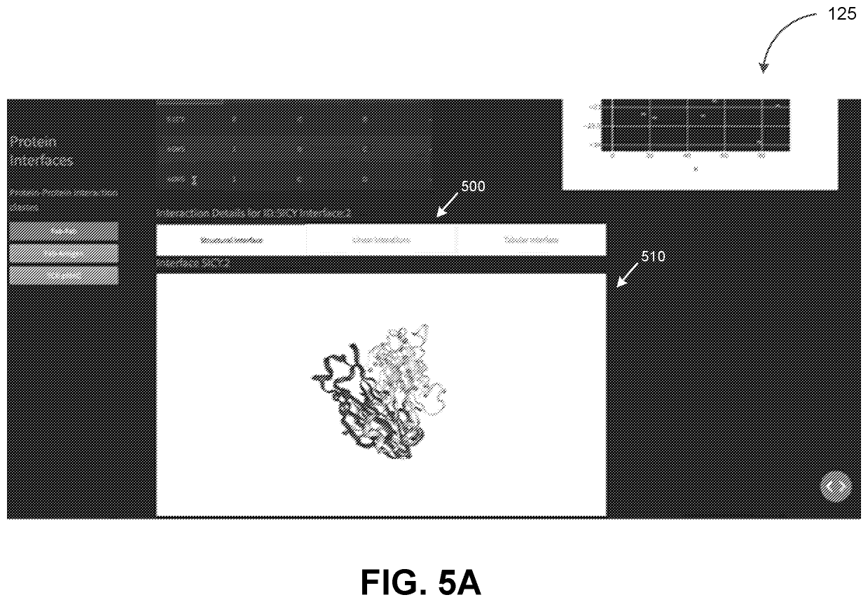

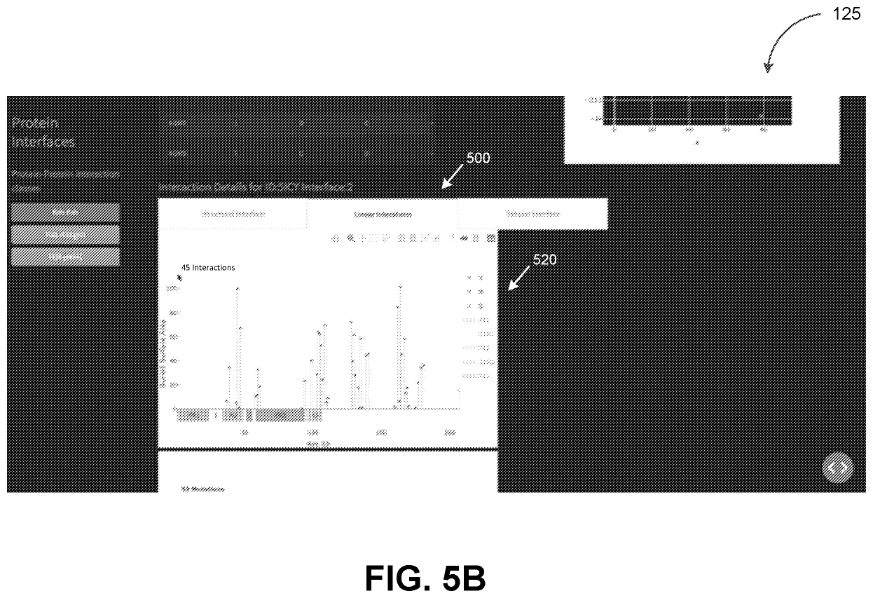

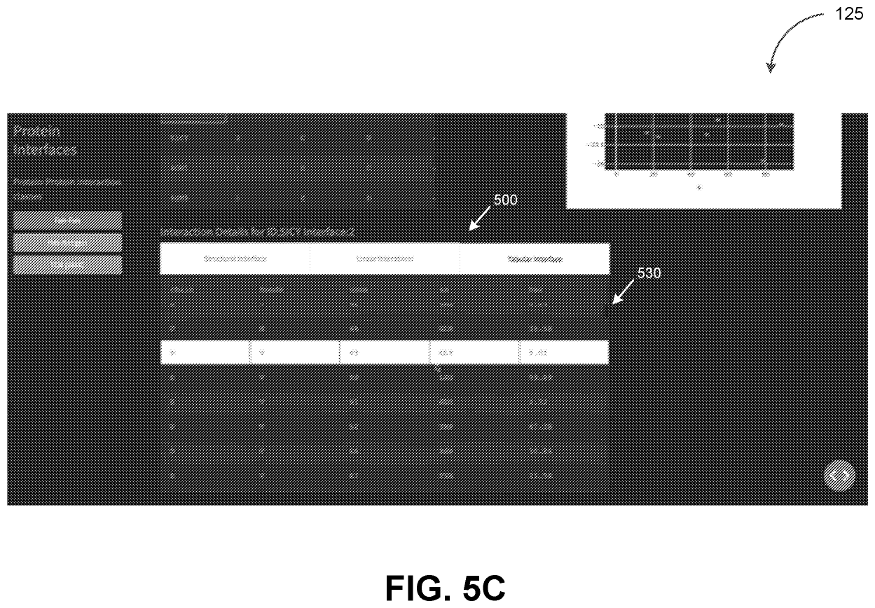

FIGS. 5A-C depict screenshots of various examples of the user interface 125 generated by the analysis engine 110 in response to the selection of the protein-to-protein interface 440 for further analysis. As shown in FIGS. 5A-C, the user interface 125 may include one or more input controls 500 for selecting between a structural representation, a linear representation, and a tabular representation of the protein-to-protein interface 440 selected for further analysis.

Referring now to FIG. 5A, the example of the user interface 125 shown in FIG. 5A includes a structural representation 510 of the protein-to-protein interface 440 selected for further analysis. The structural representation 510 may include visual indicators identifying, within the corresponding interacting protein sequences, the protein-to-protein interface 440 as well as other components of the interacting protein sequences including, for example, the heavy chain, the light chain, the framework regions (FRs), and the complementarity determining regions (CDRs), and/or the like. FIG. 5B depicts another example of the user interface 125 displaying a linear representation 520 of the protein-to-protein interface 440. As shown in FIG. 5B, the linear representation 520 of the protein-to-protein interface 440 includes, for each amino acid residue present within the protein-to-protein interface 440, a visual indication (e.g., vertical bars of different heights) of a corresponding metric such as the buried surface area. It should be appreciated that the metric displayed as a part of the linear representation 520 of the protein-to-protein interface 440 may be selected based on one or more user inputs.

As shown in FIG. 5B, the amino acid residues included in the protein-to-protein interface 440 may be distributed along the horizontal axis of the linear representation 520 in accordance to the corresponding family position identifier. As such, the linear representation 520 provides a visual indication of how the amino acid residues included in the protein-to-protein interface 440 is distributed along the length of the corresponding protein sequence. Moreover, the linear representation 520 of the protein-to-protein interface 440 includes, for each amino acid residue present within the protein-to-protein interface 440, one or more visual indications of the region on a corresponding antibody occupied by the amino acid residue. For example, the linear representation 520 of the protein-to-protein interface 440 includes one or more visual indications (e.g., different color horizontal bars) identifying the amino acid residues occupying the various framework regions and complementarity determining regions of the antibody. Furthermore, the linear representation 520 of the protein-to-protein interface 440 includes one or more visual indications (e.g., different color spots) identifying the type of bond (e.g., Van der Waal (V), hydrogen (H), or salt bridge (S)) associated with each amino acid residue.

Referring now to FIG. 5C, which depicts another example of the user interface 125 generated by the analysis engine 110 in response to the selection of the protein-to-protein interface 440 for further analysis. The example of the user interface 125 shown in FIG. 5C includes a tabular representation 530 of the protein-to-protein interface 440. The tabular representation 530 of the protein-to-protein interface 440 may enumerate, for each amino acid residue included in the protein-to-protein interface 440, various metrics and properties such as family position identifier, type of amino acid residue, and buried surface area.

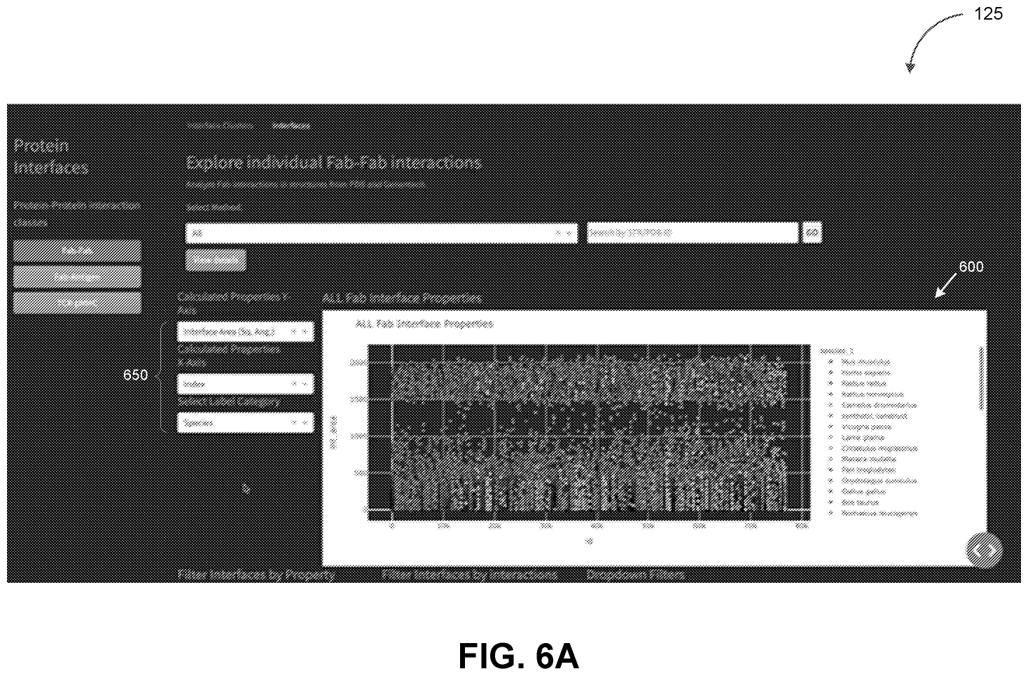

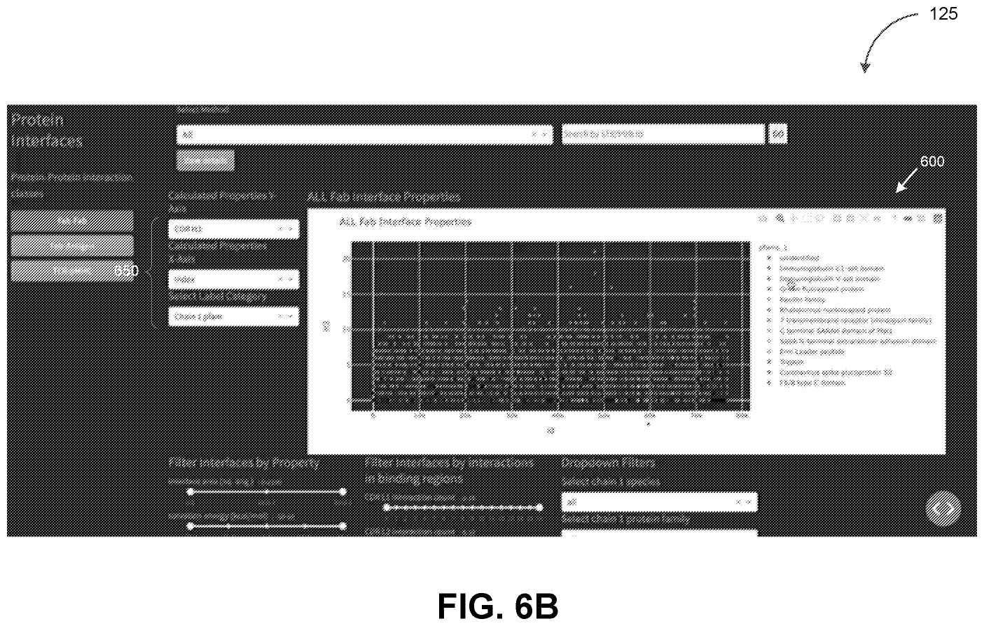

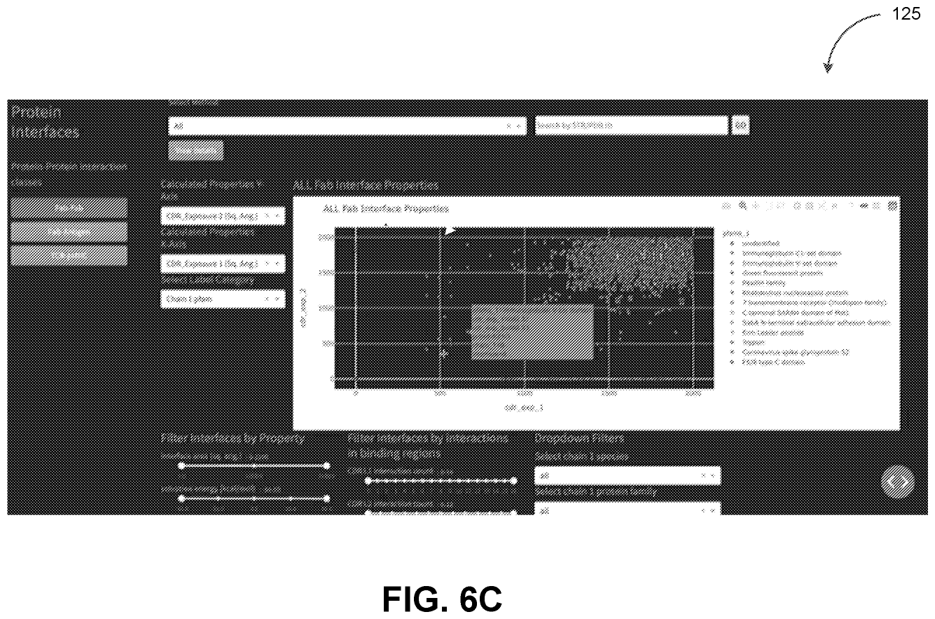

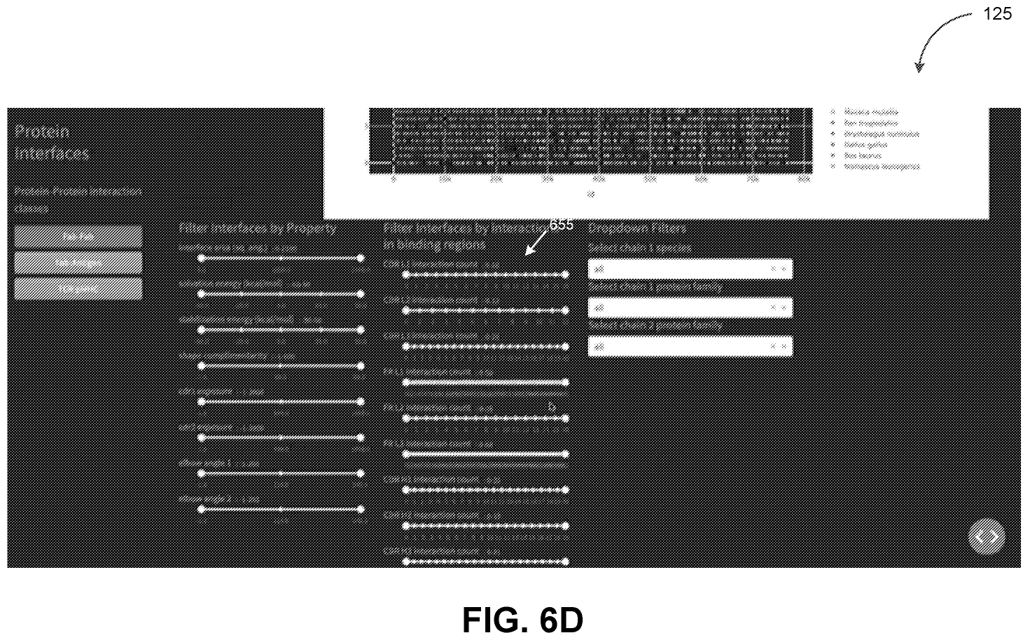

In some example embodiments, instead of and/or in addition to family level analysis and more granular analysis of specific subsets of protein-to-protein interfaces, such as the aforementioned cluster level and individual protein-to-protein interface level analysis, the analysis engine 110 may support an analysis of various supersets of protein-to-protein interfaces. FIG. 6A depicts a screenshot of another example of the user interface 125, which displays the results of the analytical workflow associated with the superset of all antigen-binding fragment (Fab) interfaces. The example of the user interface 125 shown in FIG. 6A includes a graph 600 depicting the distribution of one or more protein-to-protein interface properties across the superset of all antigen-binding fragment (Fab) interfaces. The one or more protein-to-protein interface properties depicted in the graph 600 may be selected via one or more input controls 650. For instance, FIG. 6A shows that the graph 600 may be a scatter plot depicting the distribution of interface area across the superset of all antigen-binding fragment (Fab) interfaces. Each antigen-binding fragment (Fab) interface shown in the graph 600 may be rendered using a visual indicator whose color (and/or shape) corresponds to a category label selected, for example, via the input controls 650. In the example shown in FIG. 6A, each antigen-binding fragment (Fab) interface may be rendered using a visual indicator whose color (and/or shape) corresponds to the species from which the antigen-binding fragment (Fab) interface originates. Meanwhile, in the example of the user interface 125 shown in FIG. 6B, the graph 600 may be updated to show the distribution of the length of the complementarity determining region of the antibody heavy chain (CDR-H3) across the superset of all antigen-binding fragment (Fab) interfaces. Instead of the species associated with each antigen-binding fragment (Fab) interface, each antigen-binding fragment (Fab) interface shown in the graph 600 in FIG. 6B may be rendered using a visual indicator whose color (and/or shape) corresponds to a family of the species from which the antigen-binding fragment (Fab) interface originates.

In some cases, instead of and/or in addition to depicting the distribution of a certain metric across the superset of antigen-binding fragment (Fab) interfaces, the graph 600 may be updated to display the relationship between two (or more) metrics associated with each antigen-binding fragment (Fab) interface. That is, the horizontal axis (e.g., x-axis) of the graph 600 may be updated to correspond to a first protein-to-protein interface property while the vertical axis (e.g., y-axis) of the graph 600 may be updated to correspond to a second protein-to-protein interface property. One example of this is shown in FIG. 6C where the graph 600 is updated to depict a correlation between magnitude of complementarity determining region (CDR) exposure on each protein sequence associated with the antigen-binding fragment (Fab) interface. Accordingly, as shown in FIG. 6C, the horizontal axis (e.g., x-axis) of the graph 600 may be updated to correspond to the complementarity determining region (CDR) exposure of a first protein sequence associated with each antigen-binding fragment (Fab) interface while the vertical axis (e.g., y-axis) of the graph 600 may be updated to correspond to the complementarity determining region (CDR) exposure of a second protein sequence associated with each antigen-binding fragment (Fab) interface.

FIG. 6D depicts additional input controls 655 included in the user interface 125 for adjusting one or more filters, which may be applied to select a portion of the superset of antigen-binding fragment (Fab) interfaces for inclusion in the graph 600. As shown in FIG. 6D, the portion of the superset of antigen-binding fragment (Fab) interfaces included in the graph 600 may be selected based on various properties of the antigen-binding fragment (Fab) interface such as interface area, solvation energy, stabilization energy, shape complementarity, complementarity determining region (CDR) exposures, and elbow angles. Alternatively and/or additionally, FIG. 6D shows that the portion of the superset of antigen-binding fragment (Fab) interfaces included in the graph 600 may be selected based on the number of interactions (or interacting amino acid residue) present in each region of the corresponding protein sequence such as the complementarity determining regions (CDRs) and framework regions (FRs) on the heavy chain and light chain of the corresponding antibody. It should be appreciated that the contents of the graph 600 may be updated based on the adjustments made to the one or more filters via the input controls 655.

In some example embodiments, at least a portion of the results of the analytical workflow performed by the analysis engine 110 may be applied towards one or more downstream tasks including, for example, mining the antigen-binding fragment (Fab) for stability engineering, identifying sites for antigen-binding fragment (Fab) design at the VH/VL interface, validating and generating new insights for known patterns in the variable regions of an antibody, exploring the T-cell receptor and peptide-bound major histocompatibility complexes (TCR-pMHC) interface, and generating training samples for machine learning. For example, in some instances, the analysis engine 110 may identify, based at least on the results of the analytical workflow, one or more positions within a protein sequence that may be modified or must remain fixed when designing the protein sequence. Alternatively and/or additionally, the analysis engine 110 may identify, based at least on the results of the analytical workflow, an amino acid residue that is most likely or least likely to occupy one or more positions within a protein sequence when designing the protein sequence. In the case of stability engineering, the protein-to-protein interfaces identified as a part of the analytical workflow may undergo one or more mutations to increase (or decrease) the stability of the resulting bounded complexes (e.g., between two protein molecules or between a protein molecule and a non-protein molecule). For instance, introducing mutations that improve crystal packing, hydrogen bond interactions, and/or cysteine scanning at the protein-to-protein interface on one or both molecules may create disulfide linkages, thus increasing the stability of the resulting complexes.

Referring again to FIG. 1, as another example, the analysis engine 110 may generate, based at least on the results of the analytical workflow, training data for training a machine learning model 155 at a design engine 150 to perform tasks associated with various aspects of protein design. For instance, in some cases, the analysis engine 110 may generate a starting protein sequence from one protein family (e.g., antibody, antigen-binding fragment (Fab), T-cell receptor (TCR), and/or the like) that provides a basis upon which the machine learning model 155 generates one or more protein sequences that exhibit certain desirable characteristics. This starting protein sequence may include, in cases where the desirable characteristics include binding affinity towards proteins from another protein family (e.g., another antibody, the antigen binding fragment (Fab) of another antibody, a peptide-bound major histocompatibility complex (pMHC), and/or the like), one or more amino acid residues found a threshold quantity of the protein-to-protein interfaces between the two protein families.

For example, the analysis engine 110 may determine, based at least on the results of the analytical workflow performed on the family of protein-to-protein interfaces between the two families of proteins, certain commonalities that exist across the family of protein-to-protein interfaces therebetween. These commonalities may include more than a threshold quantity of the protein-to-protein interfaces having interacting amino acid residues at certain positions (e.g., as referenced by the respective family position identifiers). In the example shown in FIG. 2, this may include family positions 17 and 29 for one protein family, which are occupied by interacting amino acid residues in more than a threshold quantity of protein-to-protein interfaces with the other protein family. Alternatively and/or additionally, the analysis engine 110 may identify the most common types and/or least common types of interacting amino acid residues present in the family of protein-to-protein interfaces between the two protein families. Accordingly, the analysis engine 110 may generate a starting protein sequence for the machine learning model 155 in which the most common positions for the protein-to-protein interface are occupied the most common types of interacting amino acid residues. In some cases, the analysis engine 110 also generate the starting protein sequence to exclude the least common types of interacting amino acid residues between the two protein families.

Alternatively and/or additionally, where the machine learning model 155 is deployed at the design engine 150 to determine one or more properties of a protein sequence, the analysis engine 110 may generate labeled training data based on the results of the analytical workflow. For example, the analysis engine 110 may generate training data that includes, for each protein sequence, one or more ground truth labels identifying various characteristics of the protein-to-protein interface associated with the protein sequence. Referring to the example shown in FIG. 2, these ground truth labels may indicate, for each protein sequence in the protein family, the positions included in the protein-to-protein interface (e.g., as referenced by family position identifiers) and the interacting amino acid residues. In some cases, the ground truth labels assigned to each protein sequence may also indicate additional characteristics of the corresponding protein-to-protein interface such as interface area, size (e.g., measured in the quantity of constituent amino acid residues), solvation energy, stabilization energy, shape complementarity, complementarity determining region (CDR) exposure, elbow angle, and/or the like. The labeled training data generated by the analysis engine 110 may be used to train the machine learning model 155 to predict, for a protein sequence from one protein family, the characteristics of the corresponding protein-to-protein interface with another protein family. These predictions may enable the design engine 150 to generate protein sequences that exhibit desirable properties and/or lack undesirable properties, particularly at the protein-to-protein interface with other protein sequences.

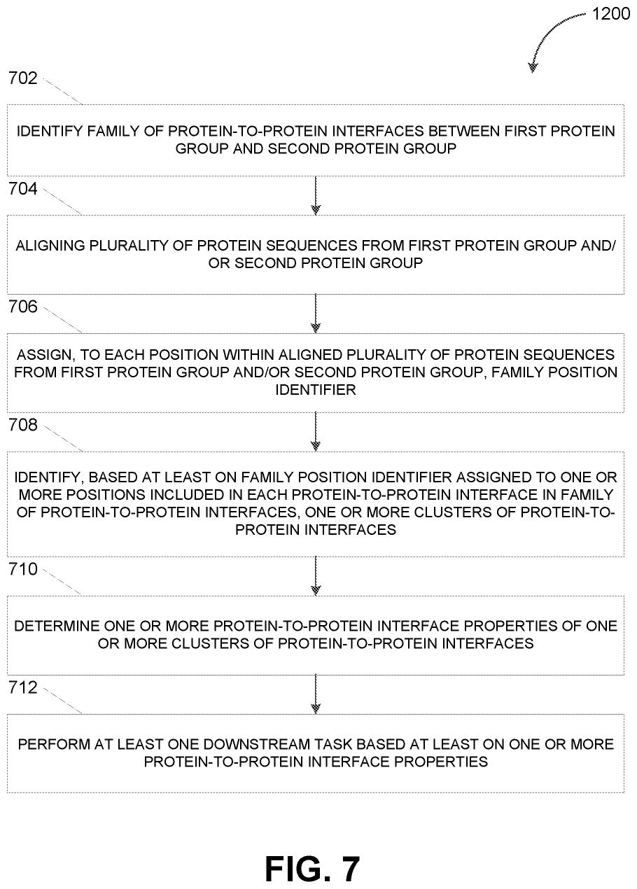

FIG. 7 depicts a flowchart illustrating an example of a process 700 for analyzing protein-to-protein interfaces, in accordance with some example embodiments. Referring to FIGS. 1-7, the process 700 may form at least a portion of an analytical workflow performed by the analysis engine 110 based on, for example, at least a portion of the protein-to-protein interface data found in the data store 130. In some cases, the analysis engine 110 may perform the process 700 in order to analyze a family of protein-to-protein interfaces, which may exist as between protein sequences from a single protein family or protein structures for two (or more) different protein families.

At 702, the analysis engine 110 may identify a family of protein-to-protein interfaces between a first protein group and a second protein group. For example, as shown in FIG. 3, the analysis engine 110 may receive, via the one or more first input controls 310 in the user interface 125, one or more user inputs selecting a family (or class) of protein-to-protein interfaces for analysis. The selected family (or class) of protein-to-protein interfaces may exist between two protein groups such as, for example, a first protein family and a second protein family. As noted, examples of protein-to-protein interface families include antigen-binding fragment to antigen-binding fragment (Fab-Fab), antigen binding fragment to antigen (Fab-Antigen), and T-cell receptor to peptide-bound major histocompatibility complexes (TCR-pMHC). Moreover, protein-to-protein interfaces may exist between protein sequences from a single protein family as well as protein sequences from two (or more) different protein families. Examples of protein families include antibodies, kinases, antigens, T-cell receptors, and peptide-bound major histocompatibility complexes (pMHCs). In some cases, the analysis engine 110 may identify, based at least on the family of protein-to-protein interfaces selected for analysis, a corresponding portion of data from the data store 130. For instance, the data store 130 may store data associated with a variety of protein-to-protein interfaces including, for a family of protein-to-protein interfaces between a first protein family and a second protein family, data corresponding to various protein-to-protein interface properties such as the positions, amino acid residues, size, area, solvation energy, stabilization energy, shape complementarity, complementarity determining region (CDR) exposure, and elbow angles associated with each protein-to-protein interface.

At 704, the analysis engine 110 may align a plurality of protein sequences from the first protein group and/or the second protein group. In some example embodiments, the analysis engine 110 may align the protein sequences from the first protein family and/or the second protein family. This alignment may include arranging the protein sequences within a protein family based on regions of similarities (e.g., same or similar subsequences of amino acid residues) present across multiple protein sequences. As noted, these regions of similarities may be attributable to the functional, structural, and/or evolutionary relationships between the protein sequences within the protein family. Moreover, a variety of sequence alignment techniques may be applied including, for example, dynamic programming, progressive or hierarchical alignment, iterative alignment, motif finding, Hidden Markov models, and/or the like.

At 706, the analysis engine 110 may assign, to each position within in an aligned plurality of protein sequences from the first protein group and/or the second protein group, a family position identifier. In some example embodiments, the analysis engine 110 may apply a universal numbering scheme, which may include assigning a family position identifier to each position in the aligned protein sequences from the first protein family and/or the second protein family. As the example in FIG. 2 shows, the same family position identifier may be assigned to a first position in a first protein sequence that is aligned with a second position within a second protein sequence even if the first position and the second position differ, thus maintaining a consistency how positions are referenced across the entire protein family. For instance, subsequent comparative analysis within the family of protein-to-protein interfaces may be performed based on the family position identifiers instead of the position identifiers applicable to individual protein sequences.

At 708, the analysis engine 110 may identify, based at least on the family position identifier assigned to one or more positions included in each protein-to-protein interface in the family of protein-to-protein interfaces, one or more clusters of protein-to-protein interfaces within the family of protein-to-protein interface. In some example embodiments, the analysis engine 110 may perform a variety of computational analysis of the family of protein-to-protein interfaces in order to identify various commonalities and/or differences present within the family of protein-to-protein interfaces. One example computational analysis is a cluster analysis in which the family of protein-to-protein interfaces undergo clustering, such as hierarchical clustering, to identify groups of similar protein-to-protein interfaces across a variety of protein-to-protein interface properties. In some cases, the analysis engine 110 may cluster the protein-to-protein interfaces based on the positions within each protein sequence, as referenced by the corresponding family position identifiers, that are involved in the interactions therebetween. Doing so may identify clusters of protein-to-protein interfaces in which the corresponding protein sequences assume a similar pose (e.g., docking pose) during interaction. Alternatively and/or additionally, the analysis engine 110 may cluster the protein-to-protein interfaces based on other protein-to-protein interface properties including, for example, protein-to-protein interface properties including, for example, shape complementarity, amino acid frequencies, complementarity determining region (CDR) lengths, antigen-binding fragment (Fab) elbow angles, interaction fingerprint (e.g., geometric features, chemical features), patch distribution, complementarity determining region (CDR) exposure, contact maps, biophysical properties (e.g., electrostatics, hydrophilicity, hydrophobicity, molecular size) of the amino acid residues, and/or the like.

At 710, the analysis engine 110 may determine one or more protein-to-protein interface properties of the one or more clusters of protein-to-protein interfaces. In some example embodiments, the analysis engine 110 may determine a variety of protein-to-protein interface properties including, for example, positions, amino acid residues, size, area, solvation energy, stabilization energy, shape complementarity, complementarity determining region (CDR) exposure, elbow angles, and/or the like. In some cases, the analysis engine 110 may determine these protein-to-protein characteristics for an entire family of protein-to-protein interfaces selected for analysis as well as a subset and/or a superset of the family of protein-to-protein interfaces selected for analysis. For instance, as the examples of the user interface 125 in FIGS. 3-4, 5A-C, and 6A-D show, the scope of the analytical workflow performed by the analysis engine 110 as well as the content displayed in the user interface 125 may be adjusted based on selections (e.g., drill-ups, drill-downs, and/or the like) made via the user interface 125. One example of the user interface 125 displaying the characteristics of an entire family of protein-to-protein interfaces is shown in FIG. 3 whereas FIG. 4 shows another example of the user interface 125 displaying the characteristics of a subset of protein-to-protein interfaces, such as a cluster of protein-to-protein interfaces from the family. An example of a further drill-down to an individual protein-to-protein interface selected from the cluster of protein-to-protein interfaces is shown in FIGS. 5A-C while an example of a drill-up to a superset of protein-to-protein interfaces is shown in FIGS. 6A-D.

At 712, the analysis engine 110 may perform at least one downstream task based on the one or more protein-to-protein interface properties. In some example embodiments, the analysis engine 110 may perform a variety of downstream tasks based on the results of the analytical workflow, which includes various protein-to-protein interface properties of the family of protein-to-protein interfaces. Examples of downstream tasks include mining the antigen-binding fragment (Fab) for stability engineering, identifying sites for antigen-binding fragment (Fab) design at the VH/VL interface, validating and generating new insights for known patterns in the variable regions of an antibody, exploring the T-cell receptor and peptide-bound major histocompatibility complexes (TCR-pMHC) interface, and generating training samples for machine learning. For example, in some instances, the analysis engine 110 may determine, based at least on the results of the analytical workflow, certain insights for designing a protein sequence that exhibits certain desirable properties, such as a binding affinity towards another protein sequence (or family of protein sequences). These insights may include one or more positions within the protein sequence that may be modified or must remain fixed when designing the protein sequence to exhibit the desirable properties. Alternatively and/or additionally, these insights may include an amino acid residue that is most likely or least likely to occupy one or more positions within the protein sequence.

In one example use case, the analysis engine 110 may perform the analytical workflow to analyze the crystal packing arrangements of 1456 antibody antigen-binding fragment (Fab) regions in the protein data bank (PDB). While a large diversity of unique protein-to-protein interfaces exists, the results of the analytical workflow indicate that certain protein-to-protein interfaces do recur with significant regularity. For example, the six most common protein-to-protein interfaces were observed in 32.2% of all antibody structures, with the most prevalent protein-to-protein interface present in 13.6% of all structures. The results of the analytical workflow also revealed certain commonalities within the protein-to-protein interfaces. For instance, the analytical workflow includes an analysis of the crystal contacts for all antigen-binding fragment (Fab) structures in the protein data bank (PDB). The results revealed recurrent packing interfaces throughout the collective antigen-binding fragment (Fab) population in the protein data bank (PDB). Thus, with this particular use case, the results of the analytical workflow provide insights into previously undiscovered oligomeric interactions between immunoglobulin domains of antibodies, thus enabling an expanded toolbox for engineering next generation biotherapeutic medicines.

In some cases, the analysis engine 110 may generate, based on the results of the analytical workflow, a starting protein sequence from one protein family (e.g., antibody, antigen-binding fragment (Fab), T-cell receptor (TCR), and/or the like) that provides a basis upon which the machine learning model 155 generates one or more protein sequences that exhibit certain desirable characteristics. Where the desirable characteristics include binding affinity towards proteins from another protein family (e.g., another antibody, the antigen binding fragment (Fab) of another antibody, a peptide-bound major histocompatibility complex (pMHC), and/or the like), this starting protein sequence may include the most common interacting amino acid residues at the most common positions for the protein-to-protein interface. Alternatively and/or additionally, where the machine learning model 155 is deployed at the design engine 150 to determine one or more properties of a protein sequence, the analysis engine 110 may generate labeled training data that includes, for each protein sequence, one or more ground truth labels identifying various characteristics of the protein-to-protein interface associated with the protein sequence. As noted, this labeled training data may be used to train the machine learning model 155 to predict, for a protein sequence from one protein family, the characteristics of the corresponding protein-to-protein interface with another protein family. Meanwhile, the design engine 150 may leverage these predictions to generate protein sequences that exhibit desirable properties and/or lack undesirable properties.

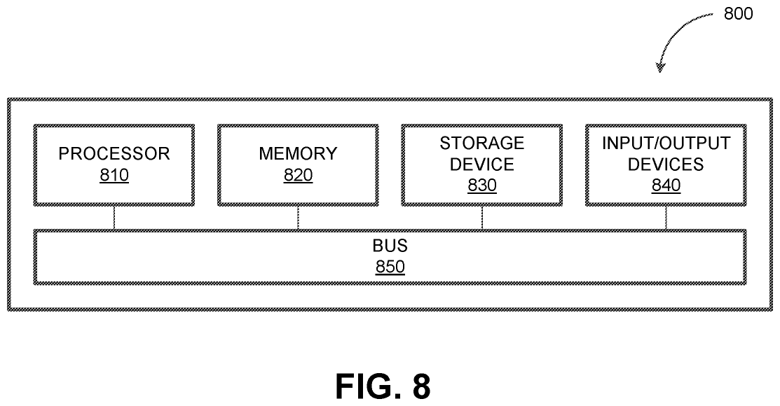

FIG. 8 depicts a block diagram illustrating an example of computing system 800, in accordance with some example embodiments. Referring to FIGS. 1 and 8, the computing system 800 may be used to implement the analysis engine 110, the client device 120, the design engine 150, and/or any components therein.

As shown in FIG. 8, the computing system 800 can include a processor 810, a memory 820, a storage device 830, and an input/output device 840. The processor 810, the memory 820, the storage device 830, and the input/output device 840 can be interconnected via a system bus 850. The processor 810 is capable of processing instructions for execution within the computing system 800. Such executed instructions can implement one or more components of, for example, the analysis engine 110, the client device 120, the design engine 150, and/or the like. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the least one data processor, cause operations comprising:
identifying a family of protein-to-protein interfaces between a first protein group and a second protein group;
assigning, to each position within an aligned plurality of protein sequences from the first protein group and/or the second protein group, a family position identifier;
identifying, based at least on the family position identifier assigned to one or more positions included in each protein-to-protein interface in a family of protein-to-protein interfaces, one or more clusters of protein-to-protein interfaces within the family of protein-to-protein interfaces; and
determining one or more protein-to-protein interface properties of the one or more clusters of protein-to-protein interfaces.

2. The system of claim 1, wherein the assigning of the family position identifier includes assigning, to a first position in a first protein sequence from the first protein group and a second position in a second protein sequence from the second protein group, a same family position identifier based at least on the first position being aligned with the second position.

3. The system of claim 1, wherein each cluster of protein-to-protein interfaces include a plurality of protein-to-protein interfaces formed by interacting protein sequences that assume a same or similar docking pose.

4. The system of claim 1, wherein the one or more clusters of protein-to-protein interfaces are identified based on at least one of a hierarchical clustering, an amino acid residue occupying the one or more positions included in each protein-to-protein interface in the family of protein-to-protein interfaces, shape complementarity, frequency of amino acid residues, complementarity determining region (CDR) length, antigen-binding fragment (Fab) elbow angle, geometric feature, chemical feature, patch distribution, complementarity determining region (CDR) exposure, contact map, and biophysical property.

5.-6. (canceled)

7. The system of claim 1, wherein the one or more clusters of protein-to-protein interfaces are identified by applying a filter imposing at least one selection criterion comprising a minimum value and/or a maximum value associated with at least one of cluster size, an interface area, a solvation energy, a stabilization energy, a shape complementarity, a complementarity determining region (CDR) exposure, and/or an elbow angle.

8. (canceled)

9. The system of claim 1, wherein the operations further comprise:
in response to a selection of a cluster of protein-to-protein interfaces from the one or more clusters of protein-to-protein interfaces, determining the one or more protein-to-protein interface properties for the selected cluster of protein-to-protein interfaces.

10. The system of claim 96, wherein the operations further comprise:
generating, for display in a user interface, a visual representation of a distribution of the one or more protein-to-protein interface properties across the selected cluster of protein-to-protein interfaces.

11. The system of claim 6, wherein the operations further comprise:
in response to a further selection of a protein-to-protein interface from the selected cluster of protein-to-protein interfaces, determining the one or more protein-to-protein interface properties for the selected protein-to-protein interface; and
generating, for display in a user interface, a structural representation of the selected protein-to-protein interface, the structural representation including a first visual indicator identifying the selected protein-to-protein interface within a first protein structure and a second protein structure associated with the selected protein-to-protein interface, the structural representation of the selected protein-to-protein interface further including a second visual indicator identifying, within the first protein structure and/or the second protein structure, one or more of a heavy chain, a light chain, a framework region (FR), and a complementarity determining region (CDR).

12.-13. (canceled)

14. The system of claim 8, wherein the operations further comprise:
generating, for display in the user interface, a linear representation of the selected protein-to-protein interface, the linear representation including one or more visual indicators identifying, for each position within the selected protein-to-protein interface, an amino acid residue occupying the position, a type of bond, and a buried surface area of the position.

15. (canceled)

16. The system of claim 1, wherein the operations further comprise:
in response to a further selection of a superset of protein-to-protein interfaces including the family of protein-to-protein interfaces, determining the one or more protein-to-protein interface properties for the selected superset of protein-to-protein interfaces; and
generating, for display in a user interface, a visual representation of a distribution of the one or more protein-to-protein interface properties across the selected superset of protein-to-protein interfaces, the visual representation includes a horizontal axis corresponding to a first protein-to-protein interface property and a vertical axis corresponding to a second protein-to-protein interface property, the visual representation further including one or more visual indicators identifying, for each protein-to-protein interface in the selected superset of protein-to-protein interfaces, an originating species and/or a family of the originating species.

17.-19. (canceled)

20. The system of claim 1, wherein the one or more protein-to-protein interface properties include shape complementarity, frequency of amino acid residues, complementarity determining region (CDR) length, antigen-binding fragment (Fab) elbow angle, geometric feature, chemical feature, patch distribution, complementarity determining region (CDR) exposure, contact map, and/or biophysical property.

21. The system of any one of claims 1 to 20, wherein the operations further comprise:
generating, for display in a user interface, a visual representation of at least a portion of the one or more protein-to-protein interface properties.

22. The system of any one of claims 1 to 21, wherein the family of protein-to-protein interfaces comprises antigen-binding fragment (Fab-Fab) interfaces, antigen binding fragment to antigen (Fab-Antigen) interfaces, or T-cell receptor to peptide-bound major histocompatibility complexes (TCR-pMHC) interfaces.

23.-24. (canceled)

25. The system of claim 1, wherein the operations further comprise:
generating, based at least on the one or more protein-to-protein interface properties, labeled training data for training a machine learning model to identify protein sequences having the one or more protein-to-protein interface properties.

26. The system of claim 1, wherein the operations further comprise:
generating, based at least on the one or more protein-to-protein interface properties, a starting protein sequence providing a basis upon which a machine learning model generates one or more additional protein sequences.

27. The system of claim 1, wherein the operations further comprise:
identifying, based at least on the one or more protein-to-protein interface properties, one or more protein-to-protein interfaces from the family of protein-to-protein interfaces; and
applying, to the one or more protein-to-protein interfaces, one or more mutations to increase a stability of a complex having the one or more protein-to-protein interface, the one or more mutations increasing the stability of the complex by improving one or more of crystal packing, hydrogen bond interactions, and cysteine scanning at the one or more protein-to-protein interface.

28. (canceled)

29. The system of claim 1, wherein the operations further comprise:
identifying, based at least on the one or more protein-to-protein interface properties, one or more positions within a protein sequence that can be modified when designing the protein sequence to exhibit one or more desirable properties.

30. The system of claim 1, wherein the operations further comprise:
identifying, based at least on the one or more protein-to-protein interface properties, one or more positions within a protein sequence that remain fixed when designing the protein sequence to exhibit one or more desirable properties.

31. The system of claim 1, wherein the operations further comprise:
identifying, based at least on the one or more protein-to-protein interface properties, an amino acid residue that is most likely or least likely to occupy at least one position within a protein sequence when designing the protein sequence to exhibit one or more desirable properties.

32. The system of claim 1, wherein the operations further comprise:
validating, based at least on the one or more protein-to-protein interface properties, one or more known patterns of amino acid residues present in the first protein group and/or the second protein group.

33.-35. (canceled)

36. A computer-implemented method, comprising:
identifying a family of protein-to-protein interfaces between a first protein group and a second protein group;
assigning, to each position within an aligned plurality of protein sequences from the first protein group and/or the second protein group, a family position identifier;
identifying, based at least on the family position identifier assigned to one or more positions included in each protein-to-protein interface in a family of protein-to-protein interfaces, one or more clusters of protein-to-protein interfaces within the family of protein-to-protein interfaces; and
determining one or more protein-to-protein interface properties of the one or more clusters of protein-to-protein interfaces.

37.-70. (canceled)

71. A non-transitory computer readable medium storing instructions, which when executed by at least one data processor, result in operations comprising:
identifying a family of protein-to-protein interfaces between a first protein group and a second protein group;
assigning, to each position within an aligned plurality of protein sequences from the first protein group and/or the second protein group, a family position identifier;
identifying, based at least on the family position identifier assigned to one or more positions included in each protein-to-protein interface in a family of protein-to-protein interfaces, one or more clusters of protein-to-protein interfaces within the family of protein-to-protein interfaces; and
determining one or more protein-to-protein interface properties of the one or more clusters of protein-to-protein interfaces.

72. (canceled)
